(19) United States
(12) Patent Application Publication
(10) Pub. No.: US 2006/0173283 A1
(43) Pub. Date: Aug. 3, 2006

(54) Method of magnetic resonance imaging

(76) Inventors: Oskar Axelsson, Malmo (SE); Klaes Golman, Malno (SE); Sven Mansson, Malmo (SE); Stefan Petersson, Malmo (SE)

Correspondence Address: GE HEALTHCARE, INC., IP DEPARTMENT, 101 CARNEGIE CENTER, PRINCETON, NJ, 08540-6231, US

(21) Appl. No.: 10/536,480

(22) PCT Filed: Nov. 27, 2003
(86) PCT No.: PCT/NO03/00395

§ 371 Date: Feb. 14, 2006

(30) Foreign Application Priority Data
Nov. 27, 2002 (SE) 200225711

(51) Int. Cl. A61B 5/05

(52) U.S. Cl. 600/420

(57) ABSTRACT

The present invention provides methods for passive visualisation of invasive devices by employing a hyperpolarised solution of a high T1 agent having a T1 value of at least 5 seconds at a field strength in the range of 0.001-5 T and a temperature in the range of 20-40° C. Devices and instruments particularly useful for being employed in the methods are also provided as well as use of the methods in imaging, surgery and therapy.

### FIELD OF THE INVENTION

The invention relates to magnetic resonance imaging (MRI) of invasive devices, e.g. during surgical and therapeutic procedures, and to devices for use with the procedures.

### DESCRIPTION OF RELATED ART

Interventional procedures in radiology are very common and increasingly popular. They are normally performed under fluoroscopic feedback by insertion of invasive instruments and/or devices like catheters, guide wires, biopsy needles, etc. through the blood vessels. The procedures can last for several hours and involve very high radiation doses for both the patient and the personnel performing them. During these procedures, it is desirable that the physician is able to locate or guide the invasive devices inserted into the patient's body when these devices are no longer visible. Since soft body parts give very little contrast on X-ray, it is often necessary to use high doses of iodinated contrast agents to localize the devices which in turn may cause problems, in particular for patients with reduced kidney function.

MRI-guided interventional procedures have gained increasing importance in recent years. Such MRI-guided procedures can be divided into two categories: intraoperative procedures, which integrate surgery with MRI, and interventional procedures for guiding, monitoring and controlling therapy. Intraoperative procedures generally require an open magnet MR imager and are desirable as the MRI can be used to define anatomy and monitor tissue function as it changes during surgery. Interventional procedures generally require only limited patient access and thus conventional, closed magnet MR imagers may be used. Normally, MRI a contrast agent or contrast medium is employed in such methods which enhances contrast and thus provides images of a higher quality. In the context of the present application, the terms “contrast agent” and “contrast medium” are used interchangeably.

The success of a MRI-guided interventional or intraoperative procedure generally depends on the ability of the MRI technique to provide accurate real time visualisation of the instruments and devices inserted into the patient's body. Generally most invasive devices are either metal based or made of polymeric materials, which is due to mechanical demands. Guide wires for example need to be stiff enough to enable them to be pushed past contortions and turns and to rotate as a rigid body all the way to the tip when the operator pushes and twists the other end, which is often a meter away from the tip of the wire. Hence guide wires are usually made of a steel core wound with a Teflon coated steel wire. However, when the device is metallic and/or conductive, it will act as a shield to the RF (radio frequency) irradiation and prevent it from reaching the contrast medium or agent within the device, thereby causing imaging defects in the region surrounding it. On the other hand, devices made of polymeric materials will not be visible in the MR image. It has been suggested to use non-conductive devices marked by incorporating bands of paramagnetic material such as for example dysprosium oxide. Such marker bands produce artefacts in the image. It has further been proposed to fill the instruments or devices with a paramagnetic contrast agent (e.g. GdDTPA) or a “blood pool” contrast agent, for example a ferromagnetic, ferrimagnetic or superparamagnetic contrast agent. The signal strength from such conventional MR contrast agents may however be inadequate for adequate “real time” tracking of the instrument. Furthermore, due to the toxicity of such agents, only limited amounts of agents may be administered or released into a living body. Another problem with this technology or, in fact, any technology that relies on proton imaging, is the massive background signal arising from water in the tissues and requiring either time consuming 3 D data acquisition or dynamic slice positioning.

U.S. Pat. No. 5,211,166 proposes visualisation of a part of an operative instrument by providing the instrument with a compound comprising a NMR active nucleus in, or in proximity to, the instrument. The NMR active nucleus can be any nucleus suitable for NM, such as the NMR active isotopes of hydrogen, phosphorus, carbon, fluorine and nitrogen. Additionally, a paramagnetic relaxant is supplied and the signals of the NMR active nuclei are amplified by dynamic nuclear polarisation (DNP) to improve the visibility of the instrument. However, it is difficult using in vivo polarisation enhancement as suggested in this document, at normal imager field strengths (>0.2 T), to achieve sufficient polarisation due to the limited penetration of the RF pulses through tissues and the instrument material, and thereby achieve a sufficient visualisation of the instrument. Another problem that occurs at higher fields is increased heating of the tissue surrounding the device. Furthermore, the amount of paramagnetic relaxant that can be released into living body tissue is also a constraint of this method due to potential toxicity of such materials.

WO-A-02/088766 proposes a method of monitoring the position of a medical instrument in vivo by introducing a hyperpolarised gas into a region within or adjacent to the instrument and imaging the hyperpolarised gas using NMR. The hyperpolarised gas can be dissolved in synthetic plasma, however only to a limited extent. ¹²⁹Xe and ³He gases are specifically mentioned. Because of their low spin density, the MR sensitivity of hyperpolarised gases is significantly lower than that of a nucleus in a soluble molecule in a suitable solvent, given that other factors such as polarisation and magnetogyric ratio are similar.

### SUMMARY OF THE INVENTION

Hence there was a need to provide improved methods for facilitating passive visualisation of invasive devices as well as a need for improved devices for use in such methods and utilisation of such methods in MR imaging, surgery and therapy. Related methods known from the prior art have drawbacks in that they expose the body under examination to high amounts of radiation, that distortions and artefacts in the MR image are unavoidable, that the contrast agents employed fail to provide the desired contrast or that they may have unacceptable toxicity at relevant doses.

It has now surprisingly been found that passive visualisation of invasive devices can be facilitated by employing a hyperpolarised solution of a high T1 agent, i.e. an agent having a T1 value of at least 5 seconds at a field strength in the range of 0.001-5 T and a temperature in the range of 20-40° C. during the time course of the visualisation procedure. Hence the invention provides methods of facilitating the visualisation of invasive devices using such hyperpolarised solution of a high T1 agent. Further, devices and instruments particularly useful for being employed in these methods are also provided as well as the use of the methods in imaging, surgery and therapy.

Thus viewed from a first aspect, the invention provides a method of facilitating the visualisation of an invasive device in a human or non-human animal body by employing a contrast medium comprising a hyperpolarised solid high T1 agent or a solution thereof having a T1 value of at least 5 seconds at a field strength of 0.001-5 T and at a temperature of 20-40° C. and MRI is used for the visualisation of the invasive device.

Viewed from a further aspect, the invention provides a method of facilitating the visualisation of an invasive device in a human or non-human animal body comprising inserting the invasive device into said body, generating an MR image of at least a part of said body containing said device and introducing a contrast medium into and optionally through said device during the time course of the visualisation procedure, characterised in that the contrast medium comprises a hyperpolarised solid or solution of a high T1 agent having a T1 value of at least 5 seconds at a field strength of 0.001-5 T and at a temperature of 20-40° C.

Viewed from a further aspect the invention provides an improved method of interventional or intraoperative MRI wherein an invasive device is inserted into a human or non-human animal body and an MR image of at least a part of said body containing said device is generated, said method comprising introducing a contrast medium into and optionally through said device during the time course of the visualisation procedure in order to facilitate visualisation of said device in said image, the improvement comprising in the contrast medium comprising a hyperpolarised solid or solution of a high T1 agent having a T1 value of at least 5 seconds at a field strength of 0.001-5 T and at a temperature of 20-40° C.

Viewed from a further aspect the invention provides the use of a hyperpolarised solid or solution of a high T1 agent having a T 1 value of at least 5 seconds at a field strength of 0.001-5 T and a temperature of 20-40° C. for the manufacture of a MR contrast medium for use in a method of diagnosis, surgery or therapy wherein an invasive device is inserted into of a human or non-human animal body, preferably into the tissue and/or vasculature, and an MR image of at least a part of said body containing said device is generated to visualise said device.

Viewed from a still further aspect the invention provides devices made of medium conductive material such as carbon fibre, e.g. carbon fibre composite material, for use in the methods according to the invention.

Further aspects of the invention will be evident from the claims and the specification:

The non-human animal body in the context of the present application can be a mammalian, avian or reptilian body. “T1” means the longitudinal relaxation time normally measured in seconds, and “T” means Tesla.

### DETAILED DESCRIPTION OF THE INVENTION

Contrast media for use in the methods of the invention comprise a hyperpolarised solid or solution of a high T1 agent, i.e. an agent having a T1 value of at least 5 seconds at a field strength of 0.001-5 T and at a temperature of 20-40° C. Such agents are well known, e.g. from WO-A-93/35508 which is hereby included by reference.

Suitable high T1 agents will comprise nuclei with non-zero nuclear spin nuclei (polarisable nuclei) preferably nuclei selected from the group consisting of ¹⁹F, ⁶Li, ¹³C, ¹⁵N, ²⁹Si and ³¹P in addition to ¹H. Particularly preferably, high T1 agents comprise nuclei selected from the group consisting of ¹³C, ¹⁵N, ¹⁹F, ²⁹Si and ³¹P nuclei, with ¹³C and ¹⁵N nuclei being particularly preferred. Other nuclei may also be used such as ⁷⁷Se, ¹¹¹Cd, ¹¹³Cd, ¹¹⁵Sn, ¹¹⁷Sn, ¹¹⁹Sn, ¹²³Te, ¹²⁵Te, ¹⁷¹ Yb, ¹⁹⁵Pt, ¹⁹⁹Hg, ²⁰³Tl, ²⁰⁵Tl and ²⁰⁷Pb, in particular for contrast media that will only be administered to the body in small amounts and concentrations or will not get into contact with the living body tissue, for example, by being held in a closed circuit inside the device during the procedure.

Preferred high T1 agents are biocompatible compounds with low toxicity. They may be used in solid form, e.g. as dispersions in a suitable solvent, but solutions of high T1 agents are preferably used, preferably in a biotolerable solvent. Particularly preferred high T1 agents are water-soluble molecules with a molecular weight below 200 D (Dalton). The contrast media for use in the method according to the invention may comprise high T1 agents comprising polarisable nuclei either in their natural abundance. More preferred, the high T1 agents are enriched in a particular position of the molecule. Preferably, high T1 agents are enriched by ¹³C and/or ¹⁵N nuclei in one or more specific positions in the molecule. In a further preferred embodiment, these enriched high T1 agents are also deuterium labelled. Preferred ¹³C enriched high T1 agents comprise one or more ¹³C nuclei which is/are surrounded by one or more non-NM active nuclei such as O, S, C or a double bond, for example a high T1 agent being ¹³C enriched at one or more carbonyl or quaternary carbons. Preferred compounds are compounds that occur naturally in the body such as amino acids, peptides, nucleic acids, carbohydrates and intermediates or metabolites occurring in the body's normal metabolic cycles and approved pharmaceuticals.

High T1 agents for use in ablation procedures include carboxylic acids and alcohols such as ethanol enriched with ¹³C. Alternatively, ablation procedures can be performed with normal carboxylic acid and alcohol such as ethanol with a minor amount of polarised compound, e.g. a ¹³C-labelled compound, added as a marker. For use in methods of therapy, suitable therapeutics preferably enriched with ¹³C and/or ¹⁵N may be used as high T1 agents, for example F-uracil and receptor targeting drugs.

Contrast media for use in the methods according to the invention preferably comprise hyperpolarised solids or solutions of high T1 agents, enriched with the mentioned nuclei and having a T1 relaxation time of 10 seconds or more, preferably 30 seconds or more, especially preferably more than 60 seconds and even more preferably more than 100 seconds at a field strength of 0.001-5 T and at a temperature of 20 to 40° C.

Methods for producing hyperpolarised high T1 agents are known, e.g. from WO-A-99/35508 and WO-A-99/24080. Preferred methods comprise the Dynamic Nuclear Polarisation (DNP) method and the Parahydrogen (PHIP) method. By using these methods it is possible to obtain hyperpolarised high T1 agents having a T1 of 5 seconds or more.

“Invasive devices” mean any invasive device and instrument used in interventional procedures such as but not limited to catheters including balloon catheters, balloons, optical fibres, guide wires, needles e.g. biopsy needles, electrodes, electrode leads, implants and stents.

If it is desired to shield the contrast medium from RF (radio frequency) irradiation until the device filled with the contrast medium reaches the region of interest (ROI), it has been found that carbon fibre-containing material such as carbon fibre composite material can be employed. For instance using a catheter comprising carbon fibre material will prevent depolarisation of the contrast medium by the RF irradiation employed during the placement of the catheter. The contrast medium will thus remain “invisible” inside the device until it is released at the ROI. Furthermore, carbon fibre material has low electrical conductivity compared to metals and will not cause substantial imaging artefacts during imaging or tissue heating. Invasive devices made of carbon fibre materials are preferably used in the methods of the invention and such devices for use in the described methods form a further aspect of the invention.

The interventional or interoperative procedure according to the invention could start with the uptake of a 2- or preferably a 3-dimensional proton image of the part of the human or non-human animal body being examined. The invasive device, e.g. a catheter, is then introduced into the vasculature, e.g. the femoral artery, or into a tissue. When it is desired to visualise the device during the procedure, the device is preferably continuously filled with contrast medium through an external duct. The signal provided by the contrast medium will allow real time visualisation and stereotactic location of the device in the 3 D proton image. The proton image of the region of interest (ROI) can be updated during the procedure, e.g. to compensate for movements. It is also possible to inject doses of contrast medium into the vasculature or tissue during the introduction of the device into the body. For example, during the introduction of a catheter into an artery, it is possible to release doses of contrast medium from the device. In this way, it is for instance possible to examine the blood flow and the blood supply to an organ in real time. When the catheter has reached the region of interest, contrast medium can be released for facilitating the interventional or interoperable procedure, for example percutaneous transluminal angiography (PTCA) with a balloon catheter optionally combined with the placement of a stent in the affected part of the artery.

The method described in the paragraph above is particularly favourable for surgical and therapeutic procedures carried out in subjects or in parts or anatomical structures of the human or non-human animal body, that are particular sensitive to radiation. Examples of such subjects are for instance pregnant women and small children, examples of such parts or anatomical structures are for instance reproductive organs. In women, about 40% of all fertility problems result from blocked fallopian tubes. The methods of the invention can therefore be utilised in procedures of examining the fallopian tubes and if necessary operating them to open such blockages interventionally by inserting a catheter through the vagina. This method forms a further aspect of the invention.

The described methods are also useful for diagnosis and precision biopsies of and surgery on solid tumours. In particular, tumours of the prostate and breasts could be sampled much more efficiently than with the currently used standard methods by using the methods according to the invention and thus simultaneously visualize soft tissue and allow precision guidance of for instance a biopsy needle.

The described methods can also be used in ablation procedures to guide the placement of clinically used devices, e.g. interstitial laser induced thermotherapy (LITT), focussed ultrasound and RF-ablation. In these procedures it is of utmost importance to place the instrument used in exactly the correct position and to follow the ablation procedure, e.g. the destruction of solid tumours. By using a method in accordance with the present invention a catheter can be positioned exactly in the right position and the vessels and surrounding tissue, as well as the tumour or structure to be removed, can be monitored during the ablation procedure.

A further aspect of the invention involves using chemicals in the ablation procedures, either alone or together with the methods described above. When the device, e.g. a catheter, is placed in the correct position either by using a RF transparent catheter with a contrast medium or using a carbon fibre catheter, the catheter can be filled with a chemical substance effective in ablation procedures which contains, or is enriched with, hyperpolarisable nuclei. Such chemical substances are for instance carboxylic acids or alcohols. Alternatively, ablation can be performed with normal carboxylic acid or alcohol, e.g. ethanol, with a minor amount of a polarised compound, e.g. a high T1 agent, as a marker. The ablation procedure can then be monitored by MRI. ¹³C or ¹⁵N enriched compounds are preferred, e.g. ¹³C enriched ethanol can be hyperpolarised and used for this purpose.

A further aspect of the invention is related to utilising the methods of the invention in therapy. As described above, a device such as a catheter can be exactly placed in the region in need of treatment and a hyperpolarisable compound, preferably a compound enriched with polarisable nuclei such as ¹³C and/or ¹⁵N, can be instilled at the site. The effect of the treatment can then be followed with MRI. Examples of compounds comprise F-uracil, e.g. ¹³C enriched and receptor targeting drugs (e.g. peptides) or bactericides, fungicides etc. The contrast medium will hence have a dual function as contrast medium and therapeutically active medium. Alternatively the therapeutically active compound can be a normal therapeutic compound containing a minor amount of polarised compound which works as a contrast medium.

Imaging parameters such as pulse sequences should be chosen carefully to achieve optimal results of the methods described above. Many methods for fast proton imaging are standardised on modern MRI scanners and it is obvious to one skilled in the art how to re-write the software to be suitable for other nuclei. Especially suitable methods are spiral and radial scan k-space sampling, optionally with sliding window update of the image. An additional option is the use of interleaved stereoscopic projections to allow for real-time stereoscopic visualisation by means of red-green stereo glasses or any of the many known stereo visualisation techniques.

The invention will be illustrated below by means of non-limiting examples and/or embodiments.

### DESCRIPTION OF THE FIGURES

FIG. 1 shows a first embodiment of an invasive device in accordance with the present invention.

FIG. 2 shows a carbon fibre tube as described and used in Example 2.

FIG. 3 shows a second embodiment of an invasive device in accordance with the present invention.

FIGS. 4a-4d show schematically cross-sections through further embodiments of invasive devices in accordance with the present invention.

FIG. 5 shows the stomach of a rat into which a guide wire has been inserted as described in Example 1.

FIG. 6 shows an X-ray angiogram of a catheter placed in the left coronary artery of a pig.

FIG. 7 shows a series of ¹³C MR images obtained during simultaneous retraction of a catheter form the left coronary artery of a pig and injection of a contrast medium comprising a hyperpolarised high T1 agent as described in Example 4

FIG. 8 shows a series of ¹³C MR images obtained during injection of a contrast medium comprising a hyperpolarised high T1 agent through a catheter inserted into the left coronary artery of a pig as described in Example 5.

FIG. 9a shows a ¹³C MR image obtained using retrospective gating during an injection of a contrast medium comprising a hyperpolarised high T1 agent through a catheter inserted into the left coronary artery of a pig as described in Example 6.

FIG. 9b shows the non-hyperpolarised proton image corresponding to FIG. 9a.

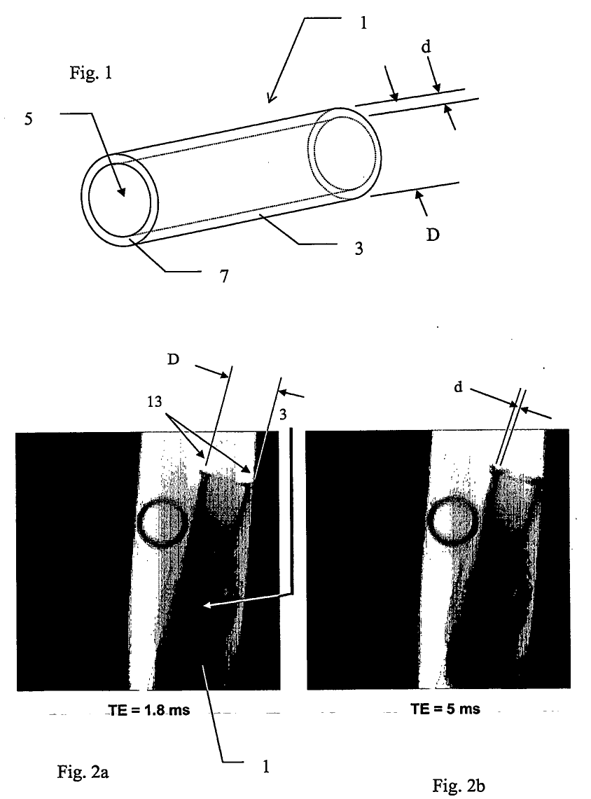

FIG. 1 shows a first embodiment of an interventional MRI invasive device (1) for use with a contrast medium comprising a hyperpolarised high T1 agent in accordance with the present invention. The invasive device (1) comprises a hollow, elongated body (3) of a diameter D mm. The body (3) has a central lumen (5) and a wall (7) of a thickness d mm. The invasive device (1) is intended to be inserted into the body of a human or non-human animal and thus may be made rigid or flexible depending on what it is used for, e.g. rigid if it is to be used as a needle and flexible if it is to be used as a catheter. The body (3) is made of carbon fibre containing material such as carbon fibre composite material, which has been found to be opaque to RF irradiation (as further described below). This is surprising because carbon fibre composite materials are normally considered to be insulating, as their electrical conductivites are almost 600 times less than that of copper.

In this embodiment, the invasive device (1) is suitable for transporting contrast medium comprising a hyperpolarised high T1 agent from a first open end, which is intended to be outside the human or non-human animal body and which is preferably connectable to a supply of contrast medium, to a second open end, which is intended to be inside or inserted into the human or non-human animal body and from which the contrast medium may be released into the ROI, for example into a blood vessel or body cavity. As the invasive device (1) is opaque to RF irradiation, the contrast medium comprising a hyperpolarised high T1 agent will not be depolarised by RF irradiation while it is shielded by the body (3).

When it is desired to visualise the devices during the entire procedure, the devices should be made of material transparent for RF (radio frequency) irradiation to provide sufficient signals from the contrast medium inside the device. Glass fibre reinforced polymer material is known to have suitable physical properties for devices such as guide wires, optical fibres, electrodes and electrode leads. Other construction polymers such as PEEK or PSU also have suitable characteristics. Needles and similar devices can be made of ceramic material such as dense zirconia, optionally coated with a polymer layer to make it less brittle or of glass fibre reinforced plastic material. Devices lacking a cavity for containing a contrast medium should be fitted with a cavity, preferably a cavity extending along the length of the device and preferably facilitating circulation of the contrast medium. Catheters may, for example, be made with double walls to allow and provide contrast medium circulation. The refill of contrast medium into the device through an external duct during the procedure will also be possible with such arrangements.

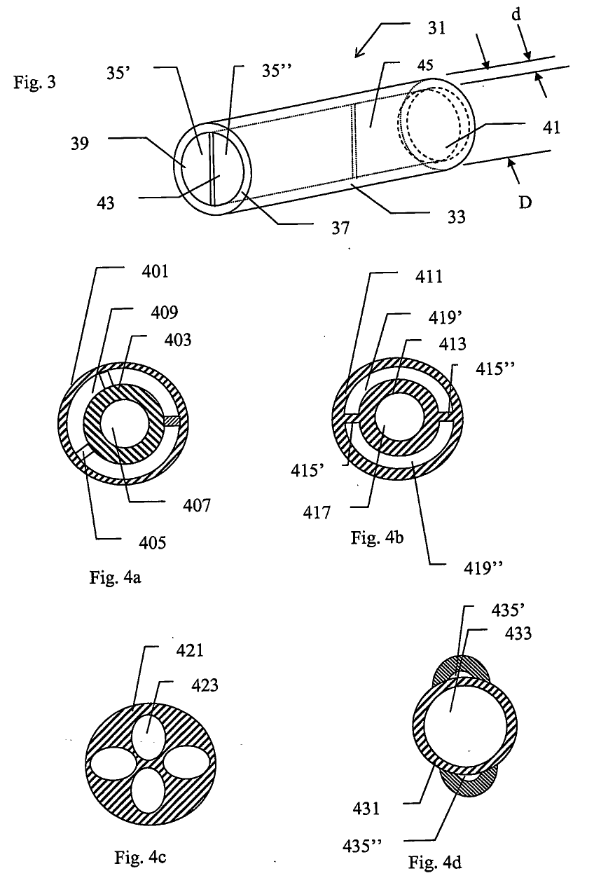

FIG. 3 shows a second embodiment of an interventional MRI invasive device (31) for use with a contrast medium comprising a high T1 agent in accordance with the present invention. The invasive device (31) comprises a hollow, elongated body (33) of a diameter D mm and a wall (37) of a thickness d mm. The body (33) comprises a first lumen (35′) and a second lumen (35″) which both extend from an open first end (39) of the body (33) to a closed second end (41) of the body (33). The first lumen 35′ and the second lumen 35″ are formed on either side of a central interior dividing wall (43) which extends from one side of the interior surface of body (33) to the, preferably diametrically, opposite side of the interior surface of body (33).

The interior dividing wall (43) does not extend all the way to the closed second end (41), thus the first (35′) and second (35″) lumen are in communication near the second end (41) of the device (31) through an opening (45) in the dividing wall (43). This allows a fluid, e.g. introduced into the first lumen (35′) at the open first end (39) to circulate along the length of the first lumen (35), then pass through the opening (45) in the dividing wall (43) near to, or at, the closed second end (41) and return via the second lumen (35″) to the open first end (39). For use in an interventional procedure, device (31) is preferably made from a material transparent to RF irradiation and a contrast medium, preferably a contrast medium comprising a hyperpolarised high T1 agent, is circulated in the invasive device (31) by being introduced though the first lumen (35′) and extracted via the second lumen (35″). In such a way new contrast medium can be introduced into the device (31) as old contrast agent is extracted from it.

In a further embodiment of the present invention, an invasive device comprising a hollow, elongated body is provided with a third lumen, which extends to the end of the device, which is intended to be introduced into a patient. This lumen can be used to accommodate a guide wire, which is used to position the device.

In another further embodiment of the present invention, an invasive device comprising a hollow, elongated body and a first and second lumen (e.g. like the device shown in FIG. 3) is provided with a third lumen, which extends to that end of the device that is intended to be introduced into the human or non-human body. This third lumen can be open at the end intended to be introduced into the human or non-human body and can be used to accommodate a tool. For example the third lumen could be provided with an inflatable balloon which can be inflated once the device has been manoeuvred to a suitable position inside the body. Alternatively the lumen could accommodate a biopsy needle, a stent or the like. An example of such a device can be seen in cross-section in FIG. 4a.

In another embodiment of the present invention, an invasive device is provided with more than three lumens, of which two may be made interconnecting to allow the circulation of a fluid, e.g. a contrast medium and the others may be used for introducing tools and/or fluids into a human or non-human body. An example of such a device can be seen in cross-section in FIG. 4c.

### EXAMPLES

The following non-limiting examples illustrate features of the invention.

### Example 1

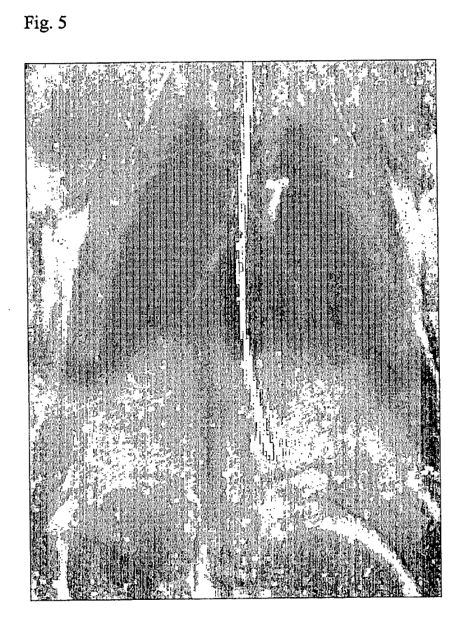

An aqueous solution of hyperpolaxised dimethylsuccinate (1-¹³C) was circulated in a closed loop moulded in a guide wire of glass-fibre reinforced plastic. The T1 of this compound under these circumstances is 70 s. The guide wire was inserted in the stomach of a rat and was imaged at the ¹³C frequency at 4 images per second. The images were colour coded and overlaid with a proton image of the rat. The guide wire is clearly visible in the rat as can be seen from the image in FIG. 5.

### Example 2

FIG. 2 shows the results of an experiment carried out using an invasive device (1) with a body (3) made from carbon fibre with a diameter D of 10 mm and a wall (7) thickness d of 1 mm. The electrical conductivity of the carbon fibre was ˜1·10⁵ S/m (c.f. copper: 5.9·10⁷ S/m). Device (1) was placed in a syringe containing water and imaged inside a 2.4 T magnet. Gradient echo images were acquired with echo time 1.8 ms as shown in FIGS. 2a and 5 ms as shown in FIG. 2b. Similar results were obtained with both echo times. Minor susceptibility artefacts (13) can be seen near the second open end of the invasive device. No signal is obtained from the lumen (3) of the of the device (except near to the second open end where radio frequency radiation has entered the lumen (3) from said second open end) showing that the wall of the invasive device is opaque to radio frequency radiation.

### Example 3

Examples of possible embodiments of invasive device cross-sections are shown in FIGS. 4a-4d.

FIG. 4a shows an invasive device formed of two concentric tubes (401, 403) with the inner tube (403) held in place in the centre of the outer tube (401) by an open system of webs (405). A first lumen (407) is formed in the interior of said inner tube and a second lumen (409) is formed by the annular space between the two tubes (401, 403).

FIG. 4b shows an invasive device formed of two concentric tubes (411,413), with the inner tube (413) held in place in the centre of the outer tube (411) by two continuous webs (415′, 415″). A first lumen (417) is formed in the interior of said inner tube (413) and a second lumen (419′) and a third lumen (419″) are formed by the semi-annular space between the two tubes (411, 413) and the webs (415′, 415″).

FIG. 4c shows an invasive device comprising an elongated body (421) with four lumens (423) formed in it.

FIG. 4d shows an invasive device comprising an elongated body (431) with a central lumen (433) and two crescent shaped lumens (435′, 435″) attached to the outer wall of said body (431).

Preferably the dimension D of the body of these devices is less than 20 mm, most preferably below 10 mm and greater than 1 mm, and the dimension d (wall thickness) of the body of said devices is preferably less than D/5 and most preferably less than D/10.

Further embodiments and modifications of invasive devices are also possible under the scope of the following claims.

### Example 4

Preparation

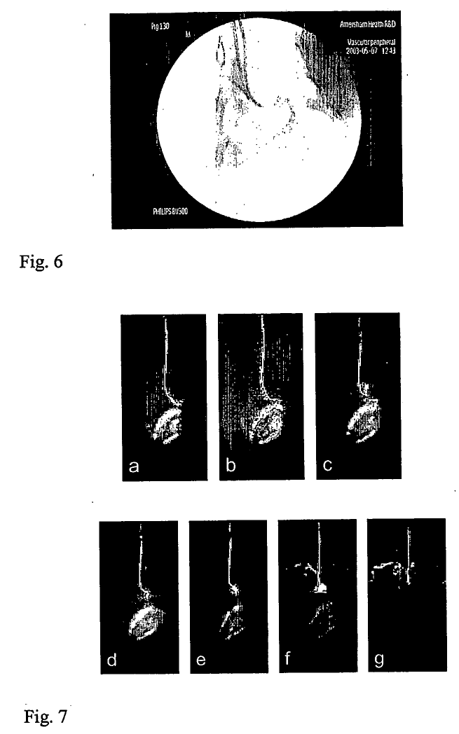

A catheter (Cobra, M, Fr.5, Terumo Europe N.V. 3001 Leuven, Belgium) was placed into the left coronary artery of a pig. Correct localisation of the catheter was confirmed by X-ray imaging using a mobile X-ray arm, a standard X-ray contrast agent was used. FIG. 6 shows the X-ray angiogram obtained. An aqueous solution of hyperpolarised hydroxyethylpropionate (1-¹³C) was prepared and used as contrast medium, the polarisation was about 28%, and the concentration of hydroxyethylpropionate (1-¹³C) was about 0.5 M. For MR imaging, a 1.5 Tesla scanner (Magnetom Sonata, Siemens Medical Solutions, Erlagen, Germany) equipped with multi nuclei extension together with a transmit/receive ¹³C coil (RapidBiomedical, Wurzburg, Germany) was used. In order to select projection angels for the FOV in question, standard proton scans were employed. ¹³C images were obtained using a fully balanced steady state pulse sequence.

Conduction of the Interventional Procedure

¹³C images (shown in FIG. 7) were acquired while the catheter was retracted from the coronary artery and at the same time the contrast medium was injected through the catheter. The following imaging parameters were used: TR/TE/FA=5.6 ms/2.8 ms/70° and FOV/Matrix=128×256 mm²/64×128. No gating was used and each image was generated in 350 ms. FIG. 6 clearly demonstrate the ability of the method of the invention to visualize a catheter during in an interventional procedure.

### Example 5

Preparation was carried out as described in Example 4.

Conduction of the Interventional Procedure

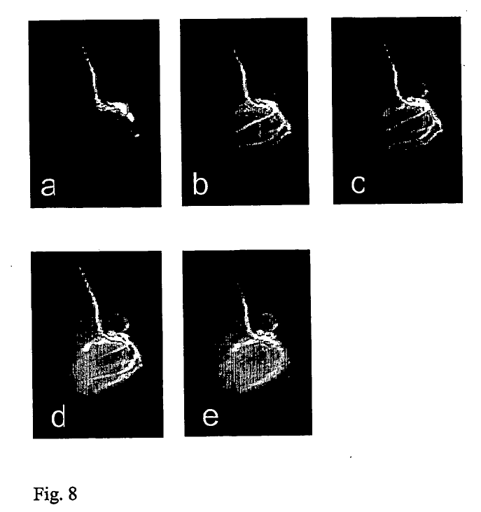

¹³C images (of which 5 extracted images are shown in FIG. 8) were acquired during injection of the contrast medium through the catheter. Cardiac gating was applied and one image was generated per heart cycle. The following imaging parameters were used: TR/TE/FA=5.1 ms/2.6 ms/70° and FOV/Matrix=1 12×256 mm²/56×128. Each image was generated in 350 ms. Image e in FIG. 8 shows the myocardium, suggesting that perfusion data may also be extracted from the imaging data in addition to angiography.

### Example 6

Preparation was carried out as described in Example 4.

Conduction of the Interventional Procedure

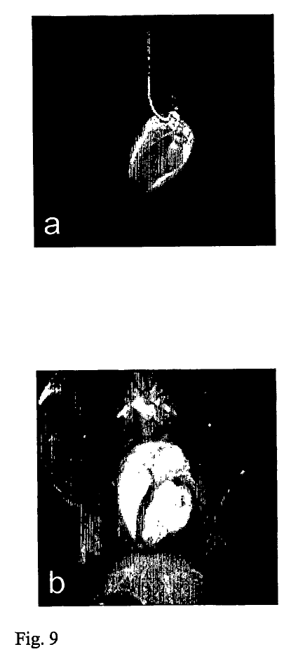

¹³C images (of which one is shown in FIG. 9a) were acquired during injection of the contrast medium through the catheter. Retrospective gating was applied. This method reorganizes the image data according to the simultaneously acquired cardiac gated signal. The cardiac cycle (systole-diastole) was divided into 22 phases and 16 were reconstructed. Imaging data from more than one heart beat were represented in each of the images of the series. For a spatial resolution of 2.0×2.0 mm² approximately 10 heart beats were needed to complete each series. The following imaging parameters were used: TR/TE/FA=5.1 ms/2.6 ms/70° and FOV/Matrix=128×256 mm²/64×128. The total scan time was 8 s. A comparison image series was acquired using standard non-hyperpolarised proton imaging. FIG. 9a shows one ¹³C image out of the series of 16 phases in the cardiac cycle. The perfusion of the cardiac muscle is evident. The corresponding non-hyperpolarised proton slice is shown in FIG. 9b.

What is claimed is:

1. A method of diagnosis, surgery or therapy wherein an invasive device is inserted into a human or non human animal body and an MR image of at least a part of said body containing said device is generated to visualise said device, comprising the step of providing an MR medium comprising a hyperpolarised solid or solution of a high T1 agent comprising nuclei selected from the group consisting of ¹⁹F, ⁶Li, ¹³C, ¹⁵N, ²⁹Si, ³¹P, ⁷⁷Se, ¹¹¹Cd, ¹¹³Cd, ¹¹⁵Sn, ¹¹⁷Sn, ¹¹⁹Sn, ¹²³Te, ¹²⁵Te, ¹⁷¹Yb, ¹⁹⁵Pt, ¹⁹⁹Hg, ²⁰³Tl, ²⁰⁵Tl and ²⁰⁷Pb and having a T1 value of at least 5 seconds at a field strength of 0.001-5 T and a temperature of 20-40° C.

2. The method as claimed in claim 1, wherein said high T1 agent comprises nuclei selected from the group consisting of ¹³C, ¹⁵N, ¹⁹F, ²⁹Si and ³¹P nuclei.

3. The method as claimed in claim 1, wherein said high T1 agent comprises nuclei selected from the group consisting of ¹³C and ¹⁵N nuclei.

4. The method as claimed in claim 1, wherein said high T1 agent has a T1 value of at least 10 seconds or more, preferably 30 seconds or more, more preferably 60 seconds or more and most preferably of more than 100 seconds at a field strength of 0.001-5 T and a temperature of 20-40° C.

5. The method as claimed in claim 1, wherein the invasive device contains a cavity for holding the contrast medium, the cavity preferably fitted with an outside duct for facilitating circulation and addition of contrast medium.

6. The method as claimed in claim 1, wherein said invasive device is made from a medium conductive material containing carbon fibre.

7. The method as claimed in claim 1, wherein the invasive device is inserted into a tissue and/or vasculature of the human or non-human animal body.

8. The method as claimed in claim 1, wherein the contrast medium additionally is a therapeutically active medium.

9. The method as claimed in claim 8 where the therapeutic active medium is instilled at the region of interest via the invasive device.

10. The method as claimed in claim 1, wherein the method is a method of examining and optionally operating the fallopian tubes.

11. The method as claimed in claim 1, wherein the method is a method for diagnosis and optional surgery on tumours.

12. The method as claimed in claim 1, wherein the method is a method for diagnosis by biopsy, preferably breast or prostate biopsy.

13. The method as claimed in claim 1, wherein the method is an ablation procedure where an additional compound effective in this ablation procedure is introduced through the invasive device.

14. A method of facilitating the visualisation of an invasive device in a human or non-human animal body comprising inserting the invasive device into said body, generating an MR image of at least a part of said body containing said device and introducing a contrast medium into and optionally through said device during the time course of the visualisation procedure, characterised in that the contrast medium comprises a hyperpolarised solid or solution of a high T1 agent comprising nuclei selected from the group consisting of ¹⁹F, ⁶Li, ¹³C, ¹⁵N, ²⁹Si, ³¹P, ⁷⁷Se, ¹¹¹Cd, ¹¹³Cd, ¹¹⁵Sn, ¹¹⁷Sn, ¹¹⁹Sn, ¹²³Te, ¹²⁵Te, ¹⁷¹Yb, ¹⁹⁵Pt, ¹⁹⁹Hg, ²⁰³Tl, ²⁰⁵Tl and ²⁰⁷Pb and having a T1 value of at least 5 seconds at a field strength of 0.001-5 T and at a temperature of 20-40° C.

15. Invasive device comprising a contrast medium comprising a hyperpolarised solid or solution of a high T1 agent comprising nuclei selected from the group consisting of ¹⁹F, ⁶Li, ¹³C, ¹⁵N, ²⁹Si, ³¹P, ⁷⁷Se, ¹¹¹Cd, ¹¹³Cd, ¹¹⁵Sn, ¹¹⁷Sn, ¹¹⁹Sn, ¹²³Te, ¹²⁵Te, ¹⁷¹Yb, ¹⁹⁵Pt, ¹⁹⁹Hg, ²⁰³Tl, ²⁰⁵Tl and ²¹⁷Pb and having a T1 value of at least 5 seconds at a field strength of 0.001-5 T and a temperature of 20-40° C., wherein said invasive device comprises a hollow elongated body made from carbon fibre containing material.

16. Invasive device according to claim 15 characterised in that the hollow elongated body is opaque to radio frequency radiation.

17. Invasive device according to claim 15 characterised in that the hollow elongated body is made of carbon-fibre composite material

18. Invasive device comprising a contrast medium comprising a hyperpolarised solid or solution of a high T1 agent comprising nuclei selected from the group consisting of ¹⁹F, ⁶Li, ¹¹³C, ¹⁵N, ²⁹Si, ³¹P, ⁷⁷Se, ¹¹¹Cd, ¹¹³Cd, ¹¹⁵Sn, ¹¹⁷ Sn, ¹¹⁹Sn, ¹²³Te, ¹²⁵Te, ¹⁷¹Yb, ¹⁹⁵Pt, ¹⁹⁹Hg, ²⁰³Tl, ²⁰⁵Tl and ²⁰⁷Pb and having a T1 value of at least 5 seconds at a field strength of 0.001-5 T and a temperature of 20-40° C., wherein said invasive device comprises a hollow elongated body with a first end and a second end, a first lumen extending from said first end to said second end and a second lumen extending from said first end to said second end, characterised in that said first lumen is in communication with said second lumen near to said second end.

19. Invasive device according to claim 18 characterised in that it comprises more than 2 lumens.

20. Invasive device according to claim 18, characterised in that the hollow elongated body is opaque to radio frequency radiation.
